(19) United States
(12) Patent Application Publication
(10) Pub. No.: US 2026/0337631 A1
(43) Pub. Date: Oct. 29, 2026

(54) USE OF SOYBEAN MYC2-like TRANSCRIPTION FACTOR GENE GmMYC3 AND KOMPETITIVE ALLELE-SPECIFIC PCR (KASP) MARKER THEREOF

(71) Applicant: NANJING AGRICULTURAL UNIVERSITY, Nanjing (CN)

(72) Inventors: Hui WANG, Nanjing (CN); Xiao LI, Nanjing (CN); Deyue YU, Nanjing (CN)

(21) Appl. No.: 19/111,480

(22) PCT Filed: Sep. 11, 2024
(86) PCT No.: PCT/CN2024/118139

§ 371 Date: Mar. 13, 2025

(30) Foreign Application Priority Data
Nov. 22, 2023 (CN) 202311563452.6

(51) Int. Cl. C12N 15/82; C12Q 1/6895

(52) U.S. Cl. CPC C12N 15/8286; C12N 15/8205; C12Q 1/6895; C12Q 2600/13; C12Q 2600/156

(57) ABSTRACT

Use of a soybean MYC2-like transcription factor gene GmMYC3 and a kompetitive allele-specific PCR (KASP) marker thereof is provided. Use of the soybean MYC2-like transcription factor gene GmMYC3 set forth in SEQ ID NO: 1 and SEQ ID NO: 2 in genetic engineering of soybean resistance to a leaf-feeding pest is provided. Use of a genetic variation on a GmMYC3 promoter set forth in SEQ ID NO: 3 and the KASP marker developed therefrom in soybean insect-resistant breeding is provided. A genotype of a deoxyribonucleotide (DNA) at 4,451,695 bp on a chromosome No. 7 in a genome of a tested soybean Glycine max Wm82.a2.v1 is detected to be CC or TT, thus determining a level for insect resistance of the tested soybean, and insect resistance of a tested soybean with a CC genotype is higher than insect resistance of a tested soybean with a TT genotype.

### CROSS REFERENCE TO RELATED APPLICATION

This patent application is a national phase of International Application No. PCT/CN2024/118139, entitled “USE OF SOYBEAN MYC2-like TRANSCRIPTION FACTOR GENE GmMYC3 AND KOMPETITIVE ALLELE-SPECIFIC PCR (KASP) MARKER THEREOF”, filed with the International Bureau on Sep. 11, 2024, which claims the benefit and priority of Chinese Patent Application No. 2023115634526 filed with the China National Intellectual Property Administration (CNIPA) on Nov. 22, 2023, the earliest priority date of this patent application is Nov. 22, 2023, and the disclosures of both are incorporated by reference herein in their entireties as parts of the present application.

### REFERENCE TO SEQUENCE LISTING

A computer readable XML file entitled “GWPCTP20241207772-Sequence Listing.xml”, that was created on Dec. 31, 2024, with a file size of about 35,498 bytes, contains the sequence listing for this application, has been filed with this application, and is hereby incorporated by reference in its entirety.

### TECHNICAL FIELD

The present disclosure relates to use of a soybean MYC2-like transcription factor gene GmMYC3 and a kompetitive allele-specific PCR (KASP) marker thereof, belonging to the technical field of genetic engineering.

### BACKGROUND

Soybean is an important food and cash crop in the world, as well as a main source of protein and oil for human beings. Spodoptera litura Fabricius is a major leaf-feeding pest in the southern and the Changjiang-Huaihe Region of China, causing serious economic losses to farmers. Spodoptera frugiperda, which has recently emerged as one of the most threatening leaf-feeding pests in maize production in Asia and Africa, can complete its life cycle in soybeans and may become a potential pest of soybeans in future maize-soybean intercropping. Therefore, breeding insect-resistant soybean varieties has become an urgent need in soybean breeding and an important task in developing sustainable agriculture.

MYC2-like transcription factors, core transcription factors in the plant jasmonic acid signaling pathway, are widely involved in various physiological processes such as regulating growth and development, stress response, and defense reaction of plants. In terms of the growth and development, Arabidopsis thaliana-derived AtMYC2/3/4 can not only act as a suppressor of flowering and stomatal development, but also regulate size, quality, and storage protein accumulation of seeds. Tomato LeMYC2 is a negative regulatory factor of blue light-mediated photomorphogenesis and can promote growth in adult tomatoes. In terms of the stress response, the Arabidopsis thaliana-derived AtMYC2 impairs salt stress tolerance in seedlings by mediating the repression of the antioxidase-derived AtCAT2. PtrMYC2 from Poncirus trifoliata promotes cold tolerance by increasing betaine biosynthesis through regulating the betaine aldehyde dehydrogenase-like of Poncirus trifoliata (PtrBADH-I). In terms of biotic stress, Arabidopsis thaliana-derived AtMYC2/3/4/5 have redundant functions in regulating glucosinolate biosynthesis, and quadruple mutants atmyc2/3/4/5 are extremely susceptible to Spodoptera exigua. Maize (Zea mays)-derived ZmMYC2a/b plays an important role in defense reactions against Mythimna separata and Spodoptera frugiperda by mediating the biosynthesis of benzoxazines and volatile terpenes. In soybean, only GmMYC1 has been reported to regulate insect resistance in tobacco. However, natural functions and regulatory mechanisms of the GmMYC family in soybean have not been reported so far.

### SUMMARY

The present disclosure provides use of a soybean MYC2-like transcription factor gene GmMYC3 in genetic engineering of soybean resistance to a leaf-feeding pest, where the soybean MYC2-like transcription factor gene GmMYC3 has a coding sequence in a resistant parent soybean KF No. 1 set forth in SEQ ID NO: 1; and the soybean MYC2-like transcription factor gene GmMYC3 has a coding sequence in a susceptible parent soybean NN 1138-2 set forth in SEQ ID NO: 2.

The present disclosure further provides a method for improving soybean resistance to a leaf-feeding pest, including: overexpressing the soybean MYC2-like transcription factor gene GmMYC3 in a soybean plant.

The present disclosure provides an expression vector for overexpressing the soybean MYC2-like transcription factor gene GmMYC3, where a plasmid of the expression vector is a pBA002 vector.

The present disclosure further provides use of an expression vector overexpressing the soybean MYC2-like transcription factor gene GmMYC3 in improving soybean resistance to a leaf-feeding pest.

The present disclosure provides a kompetitive allele-specific PCR (KASP) marker associated with soybean resistance to a leaf-feeding pest, where the KASP marker is located in a GmMYC3 promoter region at 4,451,695 bp on a chromosome No. 7 in a soybean genome Glycine max Wm82.a2.v1, where there is a T/C base mutation.

The present disclosure provides a primer combination for detecting the KASP marker associated with soybean resistance to a leaf-feeding pest, including the following primers: a forward primer F1 having a sequence set forth in SEQ ID NO: 6, a forward primer F2 having a sequence set forth in SEQ ID NO: 7, and a reverse primer R having a sequence set forth in SEQ ID NO: 8.

The present disclosure provides a kit for detecting soybean resistance to a leaf-feeding pest, including the primer combination as described above.

The present disclosure provides use of a KASP marker for the gene GmMYC3 in genetic engineering of insect-resistant soybean, the primers for the KASP marker having the following sequences: a forward primer F1 having a sequence set forth in SEQ ID NO: 6, a forward primer F2 having a sequence set forth in SEQ ID NO: 7, and a reverse primer R having a sequence set forth in SEQ ID NO: 8.

The present disclosure further provides a method for breeding a soybean variety with high resistance to a leaf-feeding pest, including: detecting a genotype of the KASP marker, then selecting a soybean variety with a CC genotype at 4,451,695 bp on a chromosome No. 7.

### BRIEF DESCRIPTION OF THE DRAWINGS

FIG. 1 shows an agarose gel electrophoresis image after PCR cloning of the gene GmMYC3; where 1 is target fragment in the resistant parent soybean KF No. 1, with a size of 1,401 bp; 2 is target fragment in the susceptible parent soybean NN 1138-2, with a size of 1,395 bp; M is Marker DL2000 Plus;

FIG. 2 shows a tissue expression pattern of the gene GmMYC3 in the resistant parent soybean KF No. 1 (n=3); where error bars represent ±SE;

FIG. 3 shows that the gene GmMYC3 can rapidly respond to the stress induction by Spodoptera litura Fabricius; where changes are shown in an expression level of the gene GmMYC3 in the resistant parent soybean KF No. 1 and the susceptible parent soybean NN 1138-2 after 0.5, 4 and 8 h of simulated insect induction (W+OS) treatment (n=3); by two-tailed t-test: **, P<0.01; ns, not significant; error bars represent ±SE;

FIG. 4 shows the subcellular localization of a GmMYC3-GFP fusion protein in Arabidopsis thaliana protoplasts; where a 35S: GFP vector is used as a control;

FIG. 5 shows the transcriptional activity assay of the full-length, N-terminal, and C-terminal of the GmMYC3 protein in yeast; where pGBKT7 (BD) vector is used as a control; -Trp indicates a nutrient-deficient medium lacking tryptophan; -Trp/-His/-Ade indicates a nutrient-deficient medium lacking tryptophan, histidine, and adenine;

FIGS. 6A-6D show PCR and qRT-PCR detection of GmMYC3-transgenic soybean; where FIG. 6A is an image showing the PCR detection of soybean overexpressing 35S:GmMYC3^{KF No. 1}; the target fragment in the resistant parent soybean KF No. 1 has a size of 1,135 bp; M is Marker DL2000 Plus; P1 is the positive plasmid pBA002-GmMYC3^{KF No. 1}; CK is the wild-type (WT); 1 to 6 are different 35S:GmMYC3^{KF No. 1} transgenic plants; FIG. 6B is an image showing the PCR detection of soybean overexpressing 35S:GmMYC3^{NN 1138-2}; the target fragment in the susceptible parent soybean NN 1138-2 has a size of 1,129 bp; M is Marker DL2000 Plus; P2 is the positive plasmid pBA002-GmMYC3^{NN 1138-2}; CK is the WT; 1 to 6 are different 35S:GmMYC3^{NN 1138-2} transgenic plants; FIG. 6C is the relative expression levels of the gene GmMYC3 in WT and GmMYC3-overexpressing soybean (n=3); by two-tailed t-test: **, P<0.01; ***, P<0.001; error bars represent ±SE; and FIG. 6D is an image showing the sequences of WT and two types of GmMYC3 homozygous mutants KO1 and KO2;

FIGS. 7A-7B show that the gene GmMYC3 positively regulates soybean resistance to Spodoptera litura Fabricius; where FIG. 7A is an image showing the size of Spodoptera litura Fabricius larvae after feeding with WT, overexpressing 35S:GmMYC3^{KF No. 1}, 35S:GmMYC3^{NN 1138-2}, and knockout (KO) transgenic soybean lines for 2 d; FIG. 7B is a diagram showing the average weight of Spodoptera litura Fabricius larvae after feeding for 2 d; where OE4 and OE28 represent two 35S:GmMYC3^{KF No. 1}-overexpressing lines, OE17, OE25, OE27, OE37, and OE38 represent five 35S:GmMYC3^{NN 1138-2}-overexpressing lines, and KO1 and KO2 represent two knockout mutants; n of WT, OE4, OE28, OE17, OE25, OE27, OE37, OE38, KO1, and KO2 soybeans were 33, 30, 33, 30, 33, 27, 33, 29, 20, and 15, respectively; by two-tailed t-test: *, P<0.05; ***, P<0.001; error bars represent ±SE;

FIGS. 8A-8B show that soybeans overexpressing the gene GmMYC3 are resistant to Spodoptera frugiperda; where FIG. 8A is an image showing the size of Spodoptera larvae after feeding with WT and overexpressing 35S:GmMYC3^{KF No. 1} OE28 soybean lines for 4 d; FIG. 8B is a diagram showing the average weight of Spodoptera frugiperda larvae after feeding for 2 d and 4 d (n=50); by two-tailed t-test: ***, P<0.001; ns, not significant; error bars represent ±SE;

FIG. 9 shows the quantitative analysis of a trypsin inhibitor content in leaves of transgenic soybeans (n=3); by two-tailed t-test: *, P<0.05; ns, not significant; error bars represent ±SE;

FIGS. 10A-10C are diagrams showing the development of a GmMYC3 KASP marker; where FIG. 10A is the genotyping of 50 soybean materials using the KASP marker; circles near the Y-axis and the triangles near the X-axis represent soybean materials carrying a T allelic variant site and soybean materials carrying a C allelic variant site, respectively, and squares near the origin represent blank controls (NTCs) without a template DNA; FIG. 10B is a diagram showing the number of soybean materials carrying a T allelic variant site and that of C allelic variant site; FIG. 10C is a diagram showing the evaluation on weight of Spodoptera litura Fabricius larvae fed with 50 soybean materials for 7 d to verify an effectiveness of the KASP marker; and error bars represent ±SE.

### DETAILED DESCRIPTION OF THE EMBODIMENTS

A purpose of the present disclosure is to provide use of a soybean MYC2-like transcription factor gene GmMYC3 in genetic engineering of insect-resistant soybean, and to provide a method for selecting a highly insect-resistant soybean variety using a KASP marker of the gene by molecular-assisted breeding.

In the present disclosure, the soybean MYC2-like transcription factor gene GmMYC3 has a coding sequence in a resistant parent soybean KF No. 1 set forth in SEQ ID NO: 1; and the soybean MYC2-like transcription factor gene GmMYC3 has a coding sequence in a susceptible parent soybean NN 1138-2 set forth in SEQ ID NO: 2.

The gene GmMYC3 is highly expressed in soybean leaves, flowers, and pods, and can quickly respond to feeding induction by the main soybean pest Spodoptera litura Fabricius. An up-regulation fold of the gene in the resistant parent soybean KF No. 1 is much greater than that in the susceptible parent soybean NN 1138-2. This gene encodes a nuclear-localized protein that displays transcriptional activation activity in yeast. Introducing respectively the gene GmMYC3 cloned from KF No. 1 and NN 1138-2 as a target gene into soybean can significantly improve the accumulation of soybean trypsin inhibitor and the resistance to Spodoptera litura Fabricius. On the contrary, knocking out the gene in soybean can weaken the insect resistance of soybean. Overexpression of the gene GmMYC3 also shows resistance to Spodoptera frugiperda, the polyphagous pest of corn and soybean. In addition, a genetic variation exists in the promoter region of this gene between the resistant and susceptible parent soybeans, and is closely linked to soybean insect resistance. Therefore, a KASP marker is developed for the genetic variation and can be used for molecular marker-assisted selection breeding.

Use of the soybean MYC2-like transcription factor gene GmMYC3 set forth in SEQ ID NO: 1 and SEQ ID NO: 2 in genetic engineering of soybean resistance to Spodoptera litura Fabricius and Spodoptera frugiperda is provided. Overexpression of the gene in soybean can significantly improve the resistance to Spodoptera litura Fabricius and Spodoptera frugiperda. Knocking out this gene in soybean can weaken resistance to Spodoptera litura Fabricius and Spodoptera frugiperda.

In the present disclosure, a method for improving soybean resistance to a leaf-feeding pest includes: overexpressing the soybean MYC2-like transcription factor gene GmMYC3 in a soybean plant. The overexpressing specifically includes: transforming a vector for overexpression into the soybean plant, where the vector for overexpression uses the soybean MYC2-like transcription factor gene GmMYC3 as a target gene. The vector for overexpression is transformed into the soybean by Agrobacterium-mediated cotyledonary node transformation in soybean. The leaf-feeding pest is one or two selected from the group consisting of Spodoptera litura Fabricius and Spodoptera frugiperda.

In the present disclosure, the expression vector for overexpressing the soybean MYC2-like transcription factor gene GmMYC3 is provided, where a plasmid of the expression vector is a pBA002 vector. The expression vector of the soybean MYC2-like transcription factor gene GmMYC3 is used to improve the soybean resistance to a leaf-feeding pest, and the leaf-feeding pest is one or two selected from the group consisting of Spodoptera litura Fabricius and Spodoptera frugiperda.

A nucleotide sequence of the genetic variation of the GmMYC3 promoter set forth in SEQ ID NO: 3 located on a soybean genome of Glycine max Wm82.a2.v1 is provided. Based on the genetic variation, a KASP marker is developed to test a genotype of a deoxyribonucleotide (DNA) at 4,451,695 bp on a chromosome No. 7 in a genome of a soybean to be tested is CC or TT, thus determining a level for insect resistance of the tested soybean, and insect resistance of a tested soybean with a CC genotype is higher than the insect resistance of a tested soybean with a TT genotype.

The present disclosure provides a KASP marker associated with soybean resistance to a leaf-feeding pest, where the KASP marker is located in a GmMYC3 promoter region at 4,451,695 bp on a chromosome No. 7 in a soybean genome of Glycine max Wm82.a2.v1, where there is a T/C base mutation.

The present disclosure provides a primer combination for detecting the KASP marker associated with soybean resistance to a leaf-feeding pest, including the following primers: a forward primer F1 having a sequence set forth in SEQ ID NO: 6, a forward primer F2 having a sequence set forth in SEQ ID NO: 7, and a reverse primer R having a sequence set forth in SEQ ID NO: 8. The present disclosure provides a kit for detecting soybean resistance to a leaf-feeding pest, including the primer combination as described above.

The present disclosure provides use of the KASP marker of the gene GmMYC3 in genetic engineering of insect-resistant breeding soybean, where there is a genetic variation of C to T substitution between a resistant parent soybean and a susceptible parent soybean in a GmMYC3 promoter region located at 4,451,695 bp on a chromosome No. 7 in a soybean genome of Glycine max Wm82.a2.v1, the genetic variation is closely linked to soybean insect resistance, and insect resistance of a tested soybean with a CC genotype is higher than insect resistance of a tested soybean with a TT genotype.

The present disclosure provides a method for breeding a soybean variety with high resistance to a leaf-feeding pest, including: detecting a genotype of the KASP marker associated with soybean resistance to a leaf-feeding pest, then selecting a soybean variety with a CC genotype at 4,451,695 bp on a chromosome No. 7. The detecting of the genotype includes: extracting a genomic DNA of a tested soybean; amplifying by real-time fluorescent PCR the genomic DNA of the tested soybean using the primer combination for testing the KASP marker associated with soybean resistance to a leaf-feeding pest; and genotyping the tested soybean according to an obtained fluorescence signal, and then determining that a tested soybean showing a 5-carboxyfluorescein (FAM) fluorescence signal is of the CC genotype. A reaction system of the real-time fluorescent PCR amplification includes: 2.5 μL of a DNA template, 2.5 μL of a V4.0 2×Kaspar mix, and 0.07 μL of the primer combination; and a reaction procedure for the real-time fluorescent PCR amplification is: activation at 94 °C for 15 min; denaturation at 94 °C for 20 sec; 10 cycles of annealing at 61 °C to 55 °C for 1 min; and 26 cycles of a process including denaturation at 94 °C for 20 sec and annealing at 55 °C for 1 min.

### Beneficial Effects

GmMYC3 encodes a key transcription factor in the plant jasmonic acid signaling pathway. Expression pattern analysis on GmMYC3 has revealed that it is mainly expressed in soybean leaves, flowers, and pod wall, and is rapidly upregulated in leaves after induction by Spodoptera litura Fabricius. Moreover, an up-regulation fold in the resistant parent soybean KF No. 1 is much greater than that in the susceptible parent soybean NN 1138-2. Subcellular localization and yeast transcriptional activity assay show that the gene GmMYC3 is a nuclear-localized transcription factor with transcriptional activity. Insect no-choice assays have confirmed that in addition to positively regulating soybean resistance to the main pest Spodoptera litura Fabricius, the GmMYC3 gene can also show resistance to the omnivorous pest of corn and soybeans, Spodoptera frugiperda. Meanwhile, overexpression of the gene GmMYC3 can increase the accumulation of a defense-related metabolite, trypsin inhibitor. Therefore, the gene GmMYC3 can be used as a target for regulating soybean resistance to Spodoptera litura Fabricius and Spodoptera frugiperda, and can be used for insect resistance engineering of soybean. A KASP marker is developed based on the genetic variation of a promoter region of the gene GmMYC3 in two parent soybean lines, can effectively distinguish resistant soybean varieties from susceptible soybean varieties, and is of great value in soybean insect-resistant breeding.

The following describes the present disclosure with reference to the accompanying drawings and examples.

Methods used in the following examples are conventional methods unless otherwise specified.

### Example 1 Analysis on Cloning, Expression Pattern, Protein Sublocalization, and Transcriptional Activation Activity of Soybean MYC2-Like Transcription Factor GmMYC3 Gene

### 1) Cloning of Soybean GmMYC3 Gene

The resistant parent soybean KF No. 1 and the susceptible parent soybean NN 1138-2 from the soybean recombinant inbred line population were used as samples, their leaves were taken, ground in a mortar and added into a 1.5 mL EP tube containing lysis solution, and the tubes were shaken thoroughly and a total RNA was extracted using an RNA extraction kit (Proteinssci Biotech Co., Ltd, Shanghai). A mass of the total RNA was identified by formaldehyde denaturing gel electrophoresis, and an RNA content was determined by spectrophotometer. The total RNA obtained was used as a template, and reverse transcription was conducted by a reverse transcription kit (TaKaRa Primer Script™ RT reagent kit) provided by TaKaRa Bio, Japan according to the instructions thereof. After obtaining a first chain of cDNA, PCR amplification was conducted with a PCR program as follows: initial denaturation at 95 °C for 3 min; 35 cycles of a process including denaturation at 95 °C for 15 s, annealing at 60 °C for 15 s, and extension at 72 °C for 90 s; incubation at 72 °C for 5 min, followed by constant-temperature treatment at 12 °C to obtain cDNAs of KF No. 1 and NN 1138-2.

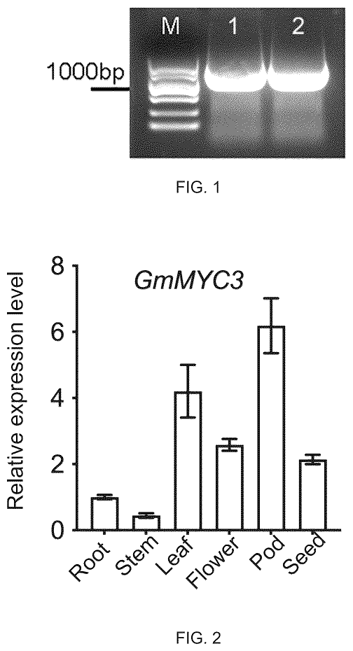

Specific primers were designed using the coding sequence of GmMYC3 (Glyma.07G051500) from a soybean database Phytozome v13 as a template, and the genes were amplified from the cDNAs of soybean parents KF No. 1 and NN 1138-2 by PCR. The obtained PCR products were respectively ligated with a T vector to construct T-GmMYC3^{KF No. 1} and T-GmMYC3^{NN 1138-2} recombinant vectors. After sequencing, complete CDS sequences of the soybean GmMYC3 gene were obtained, of which a target fragment size from KF No. 1 was 1,401 bp and a target fragment size from NN 1138-2 was 1,395 bp (FIG. 1). The gene GmMYC3 in KF No. 1 and NN 1138-2 had coding sequences set forth in SEQ ID NO: 1 and SEQ ID NO: 2, and their corresponding amino acid sequences were set forth in SEQ ID NO: 4 and SEQ ID NO: 5. The specific primer sequences required for the PCR were F: atggaggatttaatcatatctcc (SEQ ID NO: 9) and R: tcactgatccaacctcatcag (SEQ ID NO: 10).

### 2) Tissue Expression Analysis of GmMYC3

The resistant parent soybean KF No. 1 was used for tissue expression analysis of GmMYC3. Leaves, stems, and roots were sampled at the V3 stage, flowers were sampled at full bloom stage, and pod wall and seeds were sampled 15 d after anthesis. Total RNA of all samples was extracted in the same manner as that in 1), and reverse-transcribed into cDNA, and real-time RT-PCR was conducted to determine expression levels of the gene GmMYC3 in different soybean tissues. The primer sequences f fluorescence quantitative analysis of GmMYC3 were F: tggtctcgaggtcgatgtgaa (SEQ ID NO: 11) and R: gacatgctagcgtgatgaacc (SEQ ID NO: 12). A soybean Tubulin gene was used as an internal reference, and primer sequences were F: ggagttcacagaggcagag (SEQ ID NO: 13) and R: cacttacgcatcacatagca (SEQ ID NO: 14).

The results showed that GmMYC3 was highly expressed in leaves, flowers, and pod wall, but expressed at lower levels in seeds, roots, and stems (FIG. 2).

3) Expression Analysis of GmMYC3 Under Stress Induction of Spodoptera litura Fabricius

Two soybean parents, KF No. 1 and NN 1138-2, from the V3 stage were used for the induction treatment of Spodoptera litura Fabricius. Spodoptera litura Fabricius induction was simulated by wounding leaves and immediately applying oral secretions (OS) of Spodoptera litura Fabricius larvae to the wounded surface (W+OS). Total RNA was extracted from leaves of treated plants at 0.5, 4, and 8 h after W+OS treatment and control plants without induction at the same time points. The extraction of total RNA was the same as in 1). The total RNA from the leaves of the induction group and control group was used as a template for reverse transcription into cDNA to allow real-time RT-PCR. The primer sequences for GmMYC3 fluorescence quantitative primer and the primer sequences of internal reference gene Tubulin were the same as those in 2).

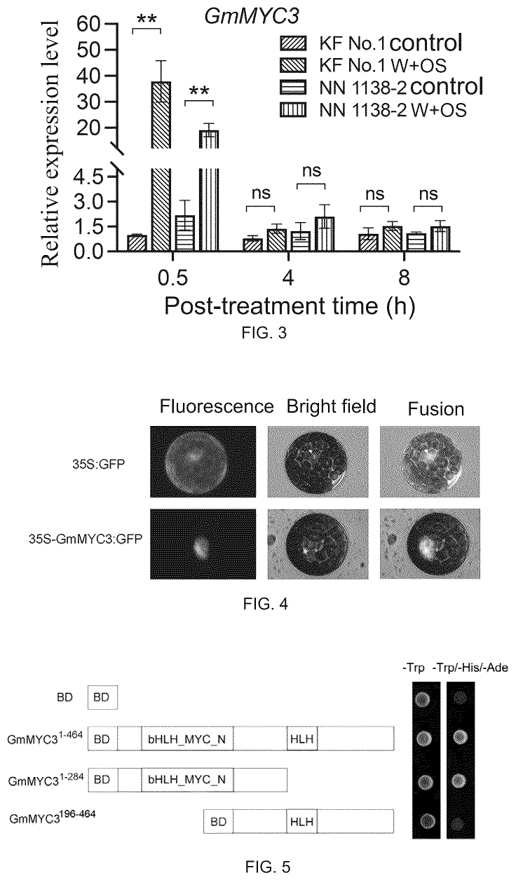

GmMYC3 could quickly respond to feeding induction by Spodoptera litura Fabricius, and up-regulation fold in the resistant parent soybean KF No. 1 was much greater than that in the susceptible parent soybean NN 1138-2 (FIG. 3).

### 4) Subcellular Localization of Soybean GmMYC3 Protein

Specific primers were designed and the coding sequence of GmMYC3 without stop codon was amplified from T-GmMYC3^{KF No. 1} vector by PCR and fused with the gene of N-terminal of green fluorescent protein (GFP) in a pAN580 vector. The vector had a 35S promoter, which could strongly induce the expression of the target gene GmMYC3 in the recipient. The fusion construct 35S:GmMYC3-GFP and the empty vector control 35S: GFP were transformed into an Arabidopsis thaliana protoplast for transient expression, and a GFP signal was observed using a laser confocal microscope. The primer sequences for constructing the vector were F: atggaggatttaatcatatctcc (SEQ ID NO: 9) and R: ctgatccaacctcatcaga (SEQ ID NO: 27).

The GFP signal showed that GmMYC3 was localized in the cell nucleus, while the fluorescent signal of the empty control was distributed throughout the cell (FIG. 4), indicating that GmMYC3 was a nuclear-localized transcription factor.

### 5) Analysis on Transcriptional Activation Activity of Soybean GmMYC3

The analysis on the transcriptional activation activity of GmMYC3 was conducted using Matchmarker GAL4 Two-Hybrid System 3 (Clontech, USA). Specific primers were designed, and the gene sequences encoding full-length, N-terminal, and C-terminal of GmMYC3 were amplified from the T-GmMYC3^{KF No. 1} vector by PCR, and fused with the GAL4 DNA BD in a pGBKT7 (BD) vector, respectively. The primer sequences for constructing the vector were F-full length: atggaggatttaatcatatctcc (SEQ ID NO: 9) and R-full length: tcactgatccaacctcatcag (SEQ ID NO: 10), F-N-terminal: atggaggatttaatcatatctcc (SEQ ID NO: 9) and R-N-terminal: cggagtctcgcgtccgagaat (SEQ ID NO: 15), and F-C-terminal: ataacccctccagatccggtt (SEQ ID NO: 16) and R-C-terminal: tcactgatccaacctcatcag (SEQ ID NO: 10). The constructs and the empty vector control BD were transformed into a yeast strain Y2HGold for plate selection, and the plates were a single-deficient medium lacking tryptophan (-Trp) and a triple-deficient medium lacking tryptophan, histidine, and adenine (-Trp/-His/-Ade), respectively.

Compared with BD used as a negative control, GmMYC3 showed transcriptional activation activity in yeast, and its transcriptional activation domain was located at the N-terminal (FIG. 5).

### Example 2 Use of Soybean MYC2-Like Transcription Factor Gene GmMYC3 in Genetic Engineering

### 1) Construction of Plant Expression Vector Carrying Soybean GmMYC3 Gene

Specific primers were designed for constructing the gene overexpression vector, and the coding sequence of GmMYC3 containing a complete ORF was amplified from the T-GmMYC3^{KF No. 1} and T-GmMYC3^{NN 1138-2} vectors by PCR, which were then inserted into the pBA002 vector under the drive of a 35S promoter to obtain recombinant vectors pBA002-GmMYC3^{KF No. 1} and pBA002-GmMYC3^{NN 1138-2}. The specific primer sequences required for the PCR were the same as those used in step 1) of Example 1. For CRISPR/Cas9 gene editing, specific primers for three target sites were designed for GmMYC3 using CRISPR-P software. The three targets were integrated into three sgDNA expression cassettes and finally ligated to a pGmUbi-Cas9-4XsgR vector (Zhang et al., Plant Biotechnology Journal, 2020, 18:1384-1395). The three pairs of target site primers were F1: ggctctcatgaacttctttg (SEQ ID NO: 17) and R1: caaagaagttcatgagagcc (SEQ ID NO: 18), F2: accaaagaggaagacgaaga (SEQ ID NO: 19) and R2: tcttcgtcttcctctttggt (SEQ ID NO: 20), and F3: acatgaacttctttgtgggaa (SEQ ID NO: 21) and R3: ttcccacaaagaagttcatgt (SEQ ID NO: 22), respectively. All constructs used for soybean transformation were introduced into an Agrobacterium tumefaciens strain EHA105.

### 2) Overexpression of GmMYC3 and Creation of Gene-Edited Soybean

A soybean cultivar GP03-8-23 was transformed using the cotyledonary node transformation. The leaf axils of soybean that had grown for 5-6 d were wounded, and the GmMYC3 overexpression and gene editing vectors obtained in step 1) were inoculated into the wounds of the soybean leaf axils and co-cultured at 25 °C for 4-5 d. Then, the cotyledonary node explants were washed with sterilized ultrapure water and Wish-Liquid, placed in SIM medium without glufosinate, and cultured at 26 °C in the light for 15 d to induce germination. After 15 d, the explants were transferred into SIM medium supplemented with 6 mg/L glufosinate. Subculture was conducted in 15-d cycles, and the dosage of glufosinate was gradually reduced. When the buds of explants grew to about 6 cm, they were transferred into a rooting medium and cultured for about 10 d to induce rooting. Transplanting was conducted once the root system had grown well.

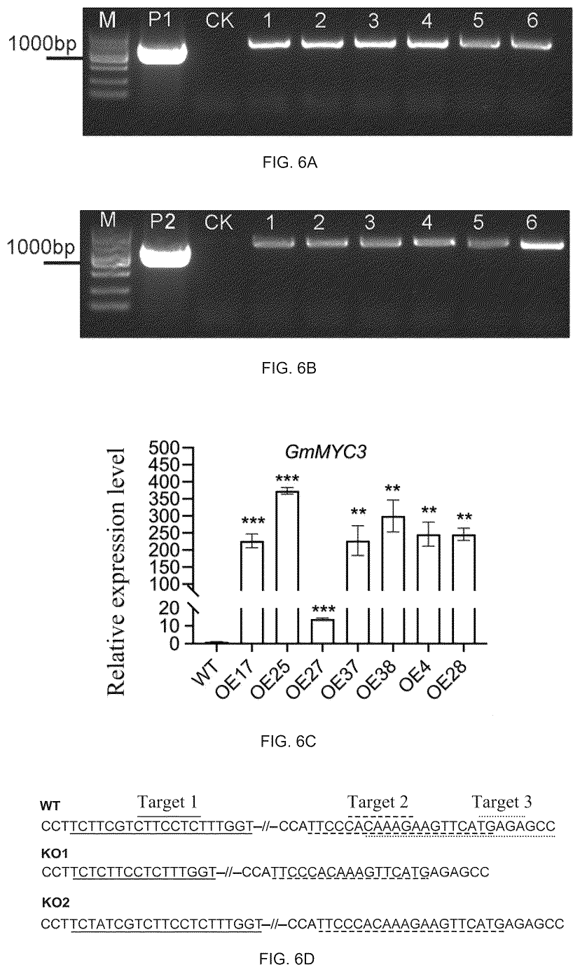

First, the soybean DNA was extracted and tested by PCR using specific primers to determine whether it was a positively transformed soybean. The primer sequences for overexpression soybean detection were F: gctcctacaaatgccatcattgc (SEQ ID NO: 23) and R: tcggttttctccctottttcttc (SEQ ID NO: 24). The images of the gel electrophoresis for tests of PCR products were shown in FIG. 6A to FIG. 6B. Real-time RT-PCR results showed that the expression levels of GmMYC3 in overexpressing soybean 35S:GmMYC3^{KF No. 1} and 35S:GmMYC3^{NN 1138-2} were both significantly increased compared with the wild-type WT (FIG. 6C). The primer sequences for fluorescence quantitative PCR of GmMYC3 and the soybean internal reference gene Tubulin were the same as those in step 2) of Example 1. For gene-edited soybeans, specific primers were designed to allow PCR amplification near the target site, and the recovered products after gel electrophoresis were sequenced and analyzed. The primer sequences for testing gene-edited soybeans were F: atcttaatccggaacccacc (SEQ ID NO: 25) and R: acttaacgtgctgtacgaggtt (SEQ ID NO: 26), and the sequencing results of the PCR products were shown in FIG. 6D.

3) Resistance of GmMYC3-Positively Regulated Soybean to Spodoptera litura Fabricius

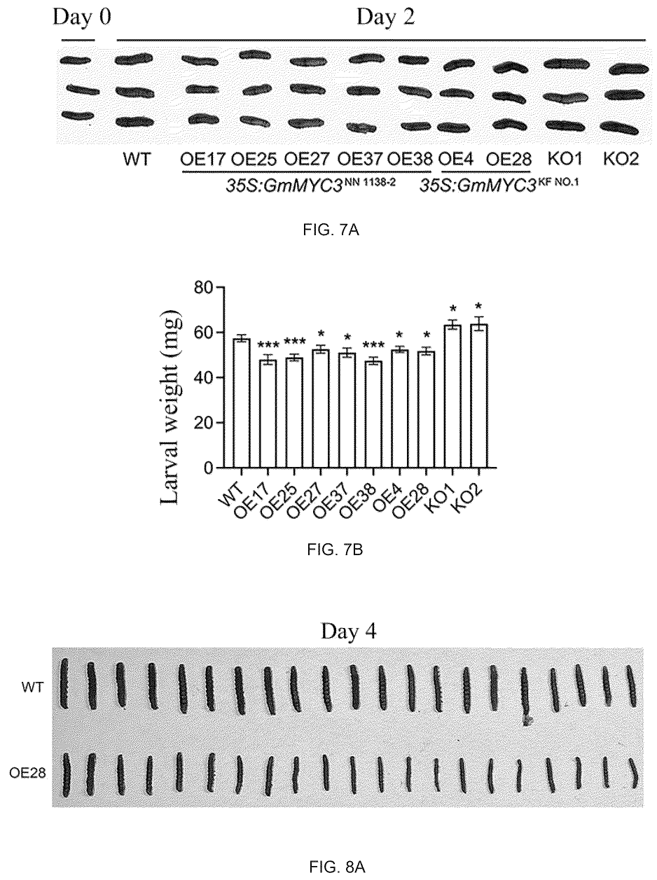

Insects were tested indoor in an air-conditioned room at 26 °C and 30% relative humidity with a photoperiod of 12 h light/12 h dark. The third-instar Spodoptera litura Fabricius larvae of uniform size were fed in 250 mL tissue culture flasks, and leaves of wild-type (WT), overexpressing 35S:GmMYC3^{KF No. 1}, 35S:GmMYC3^{NN 1138-2}, and knockout (KO) transgenic soybean lines at the full bloom stage were picked and fed to the Spodoptera litura Fabricius larvae. There were 4 larvae in each culture flask, with not less than 15 replicates for each treatment. Fresh leaves were replaced every two days, and the larvae were weighed on the day 2 of feeding. An average larval weight of each flask was calculated and used as the resistance identification index to Spodoptera litura Fabricius. Compared with the wild-type (WT), soybean lines overexpressing 35S:GmMYC3^{KF No. 1} and 35S:GmMYC3^{NN 1138-2} both significantly reduced the larval weight of Spodoptera litura Fabricius, while knockout (KO) transgenic soybean lines promoted larval growth (FIG. 7). This suggested that GmMYC3 could positively regulate soybean resistance to Spodoptera litura Fabricius, and the CDSs of GmMYC3^{KF No. 1} and GmMYC3^{NN 1138-2} might have similar resistance functions.

4) GmMYC3 Overexpression Improving Soybean Resistance to Spodoptera frugiperda

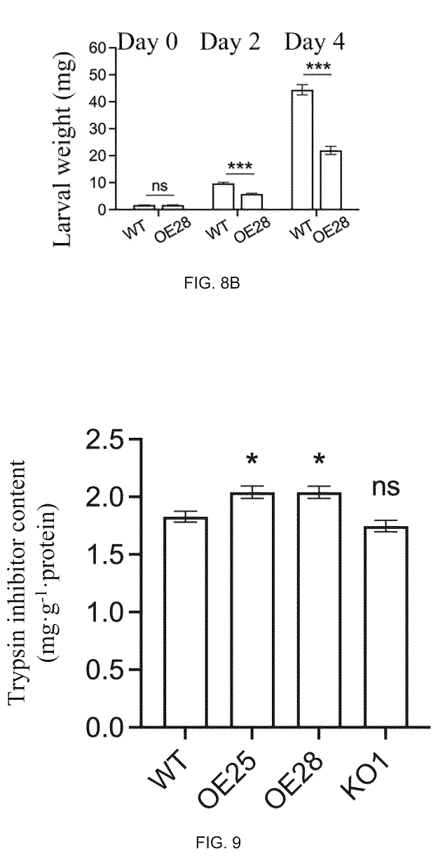

In addition to Spodoptera litura Fabricius, resistance of 35S:GmMYC3KF No. 1 OE28-overexpressing soybean to Spodoptera frugiperda was also tested indoor. Considering the cannibalistic behavior of Spodoptera frugiperda, single second-instar larvae were reared individually in 30 mL transparent plastic containers under the same conditions as those of Spodoptera litura Fabricius. The larval weight was measured on day 2 and day 4 after feeding, with 50 replicates for each treatment. Compared with the wild-type (WT), soybean overexpressing 35S:GmMYC3^{KF No. 1} OE28 significantly reduced the larval weight of Spodoptera frugiperda (FIG. 8), indicating that GmMYC3 might have a broad-spectrum resistance to leaf-feeding pests.

### 5) GmMYC3 Overexpression Promoting the Synthesis of Trypsin Inhibitor

A content of the defense metabolite trypsin inhibitor in the leaves of transgenic soybean was detected by an enzyme-linked immunosorbent assay (ELISA) kit (Nanjing Maibo Biology Science and Technology Co., Ltd.). Compared with the wild-type (WT), the trypsin inhibitor content in 35S:GmMYC3^{KF No. 1} and 35S:GmMYC3^{NN 1138-2} overexpressing soybeans was both significantly increased, while the trypsin inhibitor content in the knockout (KO) soybean was similar to that in the WT (FIG. 9). This indicated that GmMYC3 could improve soybean resistance to Spodoptera litura Fabricius and Spodoptera frugiperda by promoting the synthesis of trypsin inhibitor.

### Example 3 Development and Use of KASP Marker for the Soybean MYC2-Like Transcription Factor GmMYC3 Gene

The resistant and susceptible parent soybeans of the recombinant inbred line population underwent a C to T substitution in the GmMYC3 promoter region at 4,451,695 bp on chromosome No. 7 in genome of soybean Glycine max Wm82.a2.v1 (SEQ ID NO: 3) (https://phytozome-next.jgi.doe.gov), where a genotype of the resistant parent KF No. 1 was CC, and a genotype of the susceptible parent NN 1138-2 was TT. Targeting this genetic variation, three pairs of primers were designed using Polymarker (http://polymarker.tgac.ac.uk), including a forward primer F1: gaaggtgaccaagttcatgctGCACGAACCACTATTATCTTTTTAATCTTC (SEQ ID NO: 6), a forward primer F2: gaaggtcggagtcaacggattGCACGAACCACTATTATCTTTTTAATCTTT (SEQ ID NO: 7), and a reverse primer R: ACCAGACAGGATCAAAGATACTTT (SEQ ID NO: 8), where F1 and F2 contained FAM and HEX fluorescent linker sequences (represented by lowercase letters), respectively.

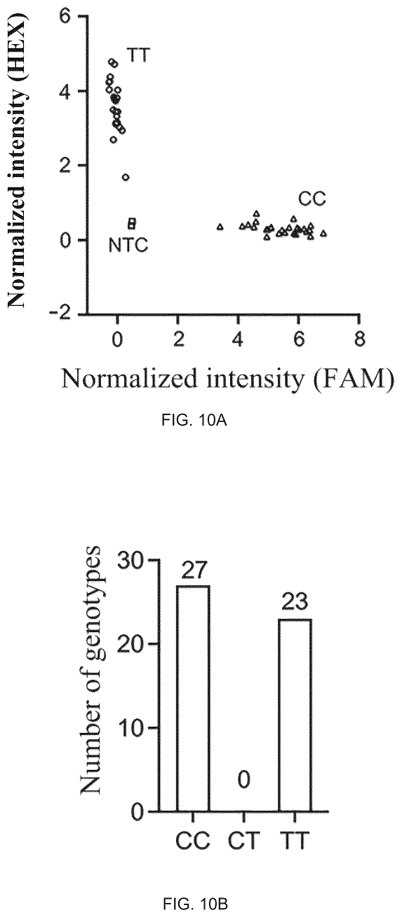

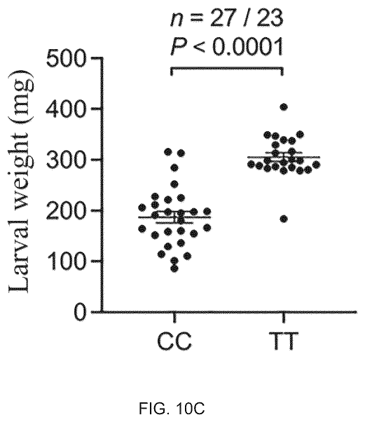

Genomic DNAs were extracted from 50 soybean materials to be tested (Table 1). Using the genomic DNA as a template, PCR amplification was conducted in an ABI9700 real-time fluorescence quantitative PCR instrument using specific primers for KASP labeling. The experiment was set up as a 5.07 μL reaction system (2.5 μL template [5-50 ng DNA], 2.5 μL V4.0 2×Kaspar mix [LGC Group], and 0.07 μL primers mixture [12 UM specific primers for each allele and 30 μM common primers]). The PCR cycle included activation at 94 °C for 15 min; denaturation at 94 °C for 20 sec, annealing at 61 °C to 55 °C for 1 min (0.6 °C reduction per cycle, 10 cycles); denaturation at 94 °C for 20 sec, annealing at 55 °C for 1 min, 26 cycles. After the PCR was completed, the instrument conducted genotyping on 50 soybean materials to be tested based on the fluorescence signal. As shown in FIG. 10A to FIG. 10C, the KASP primers could effectively separate the two genotypes, where circles close to the Y axis were the genotype harboring a T allele variation site, the genotype was TT, there were 23 materials in total, and the average weight of the Spodoptera litura Fabricius larvae fed by them was 305.23 mg; triangles close to the X axis were the genotype harboring a C allele variation site, the genotype was CC, there were 27 materials in total, and the average weight of the Spodoptera litura Fabricius larvae fed by them was 187 mg; and squares close to the origin of the XY axis were the blank control. Being consistent with the results of resistant and susceptible parent soybeans, the insect resistance of the tested parent soybeans, the resistance to the insects of the tested soybeans with CC genotype was significantly higher than that of the tested soybeans with TT genotype.

TABLE 1
Names and SNs of soybean
materials for KASP genotyping

| SN | Name |
|---|---|
| NJAU_C004 | Luhuangdou |
| NJAU_C007 | Fengxilouzihuang |
| NJAU_C010 | Wanxianbaidongdou |
| NJAU_C013 | Qingyuanxiaoqingdou |
| NJAU_C015 | Lishuizhongzihuangdou |
| NJAU_C017 | Luanchuanchengguanxiaodou |
| NJAU_C028 | Longyanqiuwudou |
| NJAU_C041 | Jianliniumaohuang |
| NJAU_C044 | Geyangqingdou |
| NJAU_C045 | Nannong73-93 |
| NJAU_C054 | Qujianghuangkengdong |
| NJAU_C058 | Liuhexiaoyeqing |
| NJAU_C067 | Anyiheidou |
| NJAU_C073 | Yunhefengheidou |
| NJAU_C074 | Feixidahuangdou |
| NJAU_C078 | Shangraowanqing |
| NJAU_C080 | Yangchunxiaoli |
| NJAU_C094 | OC7934 |
| NJAU_C096 | Dongaipingdinghuang |
| NJAU_C104 | Liaodou11 |
| NJAU_C109 | Guangshanwenzhutian |
| NJAU_C113 | heidadou |
| NJAU_C120 | Jisichanglidou |
| NJAU_C140 | York |
| NJAU_C141 | Lanchuanchengguanchunheidou |
| NJAU_C142 | Anshunbiaojiaodou |
| NJAU_C143 | Moshidadou |
| NJAU_C148 | Lichengxiaolidou |
| NJAU_C149 | Hezepingdingdou |
| NJAU_C151 | Laoshuyan |
| NJAU_C153 | Yixianheidou |
| NJAU_C155 | Huimintiezhugan |
| NJAU_C158 | Tonganyouzaidou |
| NJAU_C160 | Jingdehuangdou |
| NJAU_C162 | Hongdouzi |
| NJAU_C164 | Heiludou2hao |
| NJAU_C170 | Linheidou |
| NJAU_C172 | Hengshanhongdou |
| NJAU_C180 | Linjiangheidou |
| NJAU_C182 | Huangdou |
| NJAU_C188 | Heidadou |
| NJAU_C191 | Guangji |
| NJAU_C198 | Mianyangbaimaodou |
| NJAU_C200 | 74906-08 |
| NJAU_C201 | PI227687 |
| NJAU_C204 | Congyanghouzimao |
| NJAU_C208 | Shuyangdabaipi |
| NJAU_C215 | Bethol |
| NJAU_C218 | Wan82-178 |
| NJAU_C219 | Yudou16 |

NOTE: the above 50 soybean materials appeared in a publicly published literature (Liu, H., Che, Z., Zeng, X. et al. (2016). Identification of single nucleotide polymorphisms in soybean associated with resistance to common cutworm (Spodoptera litura Fabricius). Euphytica, 209:49-62).

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A method for genetically engineering a soybean for resistance to a leaf-feeding pest, comprising using a soybean MYC2-like transcription factor gene GmMYC3 wherein the soybean MYC2-like transcription factor gene GmMYC3 has a coding region sequence in a resistant parent soybean KF No. 1 set forth in SEQ ID NO: 1; and the soybean MYC2-like transcription factor gene GmMYC3 has a coding region sequence in a susceptible parent soybean NN 1138-2 set forth in SEQ ID NO: 2.

2. The method according to claim 1, wherein the leaf-feeding pest is one or two selected from the group consisting of Spodoptera litura Fabricius and Spodoptera frugiperda.

3. The method according to claim 1, wherein overexpressing the GmMYC3 gene improves resistance to Spodoptera litura Fabricius and Spodoptera frugiperda of the soybean.

4. The method according to claim 1, wherein knocking out the GmMYC3 gene weakens insect resistance of the soybean.

5. A method for improving soybean resistance to a leaf-feeding pest, comprising: overexpressing a soybean MYC2-like transcription factor gene GmMYC3 having a coding region sequence set forth in SEQ ID NO: 1 or SEQ ID NO: 2 in a soybean plant.

6. The method according to claim 5, wherein the leaf-feeding pest is one or two selected from the group consisting of Spodoptera litura Fabricius and Spodoptera frugiperda.

7. The method according to claim 5, wherein the overexpressing specifically comprises: transforming an overexpression vector into the soybean plant, wherein the overexpression vector contains the soybean MYC2-like transcription factor gene GmMYC3 as a target gene.

8. The method according to claim 7, wherein the overexpression vector is transformed by an Agrobacterium-mediated cotyledonary node transformation method.

9-10. (canceled)

11. The method according to claim 5, wherein the leaf-feeding pest is one or two selected from the group consisting of Spodoptera litura Fabricius and Spodoptera frugiperda.

12. A kompetitive allele-specific PCR (KASP) marker associated with soybean resistance to a leaf-feeding pest, wherein the KASP marker is located in a GmMYC3 promoter region at 4,451,695 bp on a chromosome No. 7 in a soybean genome Glycine max Wm82.a2.v1, where there is a T/C base mutation.

13. A primer combination for detecting the KASP marker according to claim 12, comprising: a forward primer F1 having a sequence set forth in SEQ ID NO: 6, a forward primer F2 having a sequence set forth in SEQ ID NO: 7, and a reverse primer R having a sequence set forth in SEQ ID NO: 8.

14. A kit for detecting soybean resistance to a leaf-feeding pest, comprising the primer combination according to claim 13.

15. A method for breeding an insect-resistant soybean, comprising using a KASP marker for a soybean MYC2-like transcription factor gene GmMYC3 having a coding region sequence set forth in SEQ ID NO: 1 or SEQ ID NO: 2, wherein a primer combination for detecting the KASP marker is the primer combination according to claim 13.

16. The method according to claim 15, wherein there is a genetic variation of C to T substitution between a resistant parent soybean and a susceptible parent soybean in a GmMYC3 promoter region located at 4,451,695 bp on a chromosome No. 7 in a soybean genome Glycine max Wm82.a2.v1, the genetic variation is closely linked to soybean insect resistance, and the insect resistance of a tested soybean with a CC genotype is higher than the insect resistance of a tested soybean with a TT genotype.

17. A method for breeding a soybean variety with high resistance to a leaf-feeding pest, comprising: detecting a genotype of the KASP marker according to claim 12, and selecting a soybean variety with a CC genotype at 4,451,695 bp on a chromosome No. 7.

18. The method according to claim 17, wherein the detecting a genotype specifically comprises:
extracting a genomic DNA of a tested soybean;
amplifying the genomic DNA of the tested soybean by real-time fluorescent PCR using the primer combination according to claim 13; and
genotyping the tested soybean based on an obtained fluorescence signal, and those showing a 5-carboxyfluorescein (FAM) fluorescence signal are of CC genotypes.

19. The method according to claim 18, wherein a reaction system of the real-time fluorescent PCR amplification comprises: 2.5 μL of a DNA template, 2.5 μL of a V4.0 2×Kaspar mix, and 0.07 μL of the primer combination.

20. The method according to claim 18, wherein a reaction procedure of the real-time fluorescent PCR amplification comprises: activation at 94 °C for 15 min; denaturation at 94 °C for 20 sec; annealing at 61 °C to 55 °C for 1 min, 10 cycles; and 26 cycles of a process comprising denaturation at 94 °C for 20 sec and annealing at 55 °C for 1 min.


SEQUENCE LISTING (27 sequences)

SEQ ID NO: 1 - DNA, 1401 bp, Glycine max
  source 1..1401: mol_type = genomic DNA; note = coding sequence for GmMYC3 of Glycine max KF No. 1, a parent soybean resistant to leaf-feeding pests
atggaggatttaatcatatctccttcttcgtcttcctctgtggtctctctaccccatgaaaacccaacaccaacaccaacaccaacccttctacaaaaactccaattcctcctccaaagccaacccgattggtgggtctacgccattttctggcaagcctcacacgacgacaatggcaacctctacttatccttcggagagggccatttccaaggcaccaaagaaacttccccaaagtcactaaccattcccacaaagaagttcatgagagcccccacaaacgacaccaacaacatcaacgacgccgagtggttctacgtcgtgtcgttgactcgcaccttcgcagtcaacaataatgcttcttcttcttcttcttctctcccgggaaagtcttttgctttaggctcggttctctggctcaacaacatgcacgagctccaattctacaactgcgagagatcaaacgaggcgcaagtgcacgggatcgaaacgttgatctgcatcccgacacaaaatggcgtcgtcgaaatgggatcctacgacaccatcaaacaaaactggaacctcgtacagcacgttaagtctctcttcataacccctccagatccggttcctgtcgaaatcctcgacgaccacaccatctccttcgctgacatcggcatcgttgccggagtccaagaaaccaaaaagcggagaataaaccaaacacaaacacaaaaacctcccaggaaaaacgacaactatgtcaactccgaacactccgattcggattgtcccacgttgccaaccgccaccaccccgacaacctcggagccgaagaaaagagggagaaaaccgattctcggacgcgagactccggtaaaccacgtggaggcagagagacaacgtagggagaagctcaatcatcgattctatgcgcttcgtgctgttgttccgaatgtttcgaggatggacaaggcttcgctgttgtcggacgctgtcgcctacatcagcgagctgaaggcgaagattgagtacttagagtcgcagcagccgagagacagtagtaaaaaagtgaagacagaaatgacggatactctagataaccatagcaccacgacaatatcgacagtggtggatcaaagcgggcctgaacctagattgggcccaagcccgcttggtctcgaggtcgatgtgaaaatcgtggggcccgatgccatggttagggtacaatcagaaaatgtgaaccaccctggggcgaggctaatgggtgctttgagagacctagaatttcaggttcatcacgctagcatgtcttgtgttaacgatctcatgcttcaagatgtggttgttaagcttcccaatggaatgaggagtgaagagggtcttaaatctgcgattctgatgaggttggatcagtga

SEQ ID NO: 2 - DNA, 1395 bp, Glycine max
  source 1..1395: mol_type = genomic DNA; note = coding sequence for GmMYC3 of Glycine max NN 1138-2, a parent soybean susceptible to leaf-feeding pests
atggaggatttaatcatatctccttcttcgtcttcctctttggtctctctaccccatgaaaacccaacaccaacaccaacccttctacaaaaactccaattcctcctccaaagccaacccgattggtgggtctacgccattttctggcaagcctcacacgacgacaatggcaacctctacttatccttcggagagggccatttccaaggcaccaaagaaacttccccaaagtcactaaccattcccacaaagaagttcatgagagcccccacaaacgacaccaacaacatcaacgacgccgagtggttctacgtcgtgtcgttgactcgcaccttcgcagtcaacaataatgcttcttcttcttcttcttctctcccgggaaagtcttttgctttaggctcggttctctggctcaacaacatgcacgagctccaattctacaactgcgagagatcaaacgaggcgcaagtgcacgggatcgaaacgttgatctgcatcccgacacaaaatggcgtcgtcgaaatgggatcctacgacaccatcaaacaaaactggaacctcgtacagcacgttaagtctctcttcataacccctccagatccggttcctgtcgaaatcctcgacgaccacaccatctccttcgccgacatcggcatcgttgccggagtccaagaaaccaaaaagcggagaataaaccaaacacaaacacaaaaacctcccaggaaaaacgacaactatgtcaactccgaacactccgattcggattgtcccacgttgccaaccgccaccaccccgacaacctcggagccgaagaaaagagggagaaaaccgattctcggacgcgagactccggtaaaccacgtggaggcagagagacaacgtagggagaagctcaatcatcgattctatgcgcttcgtgctgttgttccgaatgtttcgaggatggacaaggcttcgctgttgtcggacgctgtcgcctacatcagcgagctgaaggcgaagattgagtacttagagtcgcagcagccgagagacagtagtaaaaaagtgaagacagaaatgacggatactctagataaccatagcaccacgacaatatcgacagtggtggatcaaagcgggcctgaacctagattgggcccaagcccgcttggtctcgaggtcgatgtgaaaatcgtggggcccgatgccatggttagggtacaatcagaaaatgtgaaccaccctggggcgaggctaatgggtgctttgagagacctagaatttcaggttcatcacgctagcatgtcttgtgttaacgatctcatgcttcaagatgtggttgttaagcttcccaatggaatgaggagtgaagagggtcttaaatctgcgattctgatgaggttggatcagtga

SEQ ID NO: 3 - DNA, 460 bp, Glycine max
  source 1..460: mol_type = genomic DNA; note = a nucleotide sequence of the genetic variation of the GmMYC3 promoter located on a soybean genome of Glycine max Wm82.a2.v1
tcataaatatgttcaattttttcactttctaactcaactttttatctcgcagttactcatttccccaccaaaatttaacacttctgcccccctaccatataacccacacacttctgccccccctaccatataaccccccgtcccgaataaatatgacaatgggacggggcacgaaccactattatctttttaatcttygactctgattctttaatatgtttttatatttatacttgatatttagtggattaaaatttatttattatcttatttttgtattcataaggtataaagtatctttgatcctgtctggtatttgatttaaattgaaaaaataatttttttaaagaaaatattaaaaatttgattttaaaaaaaataaattaattgttaaattttaactacttatatatcaataaatttattataaaacatatatctataaaatcgttaagaaaag

SEQ ID NO: 4 - protein, 466 aa, Glycine max
  source 1..466: note = amino acid sequence coded by GmMYC3 gene of Glycine max KF No. 1
MEDLIISPSSSSSVVSLPHENPTPTPTPTLLQKLQFLLQSQPDWWVYAIFWQASHDDNGNLYLSFGEGHFQGTKETSPKSLTIPTKKFMRAPTNDTNNINDAEWFYVVSLTRTFAVNNNASSSSSSLPGKSFALGSVLWLNNMHELQFYNCERSNEAQVHGIETLICIPTQNGVVEMGSYDTIKQNWNLVQHVKSLFITPPDPVPVEILDDHTISFADIGIVAGVQETKKRRINQTQTQKPPRKNDNYVNSEHSDSDCPTLPTATTPTTSEPKKRGRKPILGRETPVNHVEAERQRREKLNHRFYALRAVVPNVSRMDKASLLSDAVAYISELKAKIEYLESQQPRDSSKKVKTEMTDTLDNHSTTTISTVVDQSGPEPRLGPSPLGLEVDVKIVGPDAMVRVQSENVNHPGARLMGALRDLEFQVHHASMSCVNDLMLQDVVVKLPNGMRSEEGLKSAILMRLDQ

SEQ ID NO: 5 - protein, 464 aa, Glycine max
  source 1..464: note = amino acid sequence coded by GmMYC3 gene of Glycine max NN 1138-2
MEDLIISPSSSSSLVSLPHENPTPTPTLLQKLQFLLQSQPDWWVYAIFWQASHDDNGNLYLSFGEGHFQGTKETSPKSLTIPTKKFMRAPTNDTNNINDAEWFYVVSLTRTFAVNNNASSSSSSLPGKSFALGSVLWLNNMHELQFYNCERSNEAQVHGIETLICIPTQNGVVEMGSYDTIKQNWNLVQHVKSLFITPPDPVPVEILDDHTISFADIGIVAGVQETKKRRINQTQTQKPPRKNDNYVNSEHSDSDCPTLPTATTPTTSEPKKRGRKPILGRETPVNHVEAERQRREKLNHRFYALRAVVPNVSRMDKASLLSDAVAYISELKAKIEYLESQQPRDSSKKVKTEMTDTLDNHSTTTISTVVDQSGPEPRLGPSPLGLEVDVKIVGPDAMVRVQSENVNHPGARLMGALRDLEFQVHHASMSCVNDLMLQDVVVKLPNGMRSEEGLKSAILMRLDQ

SEQ ID NO: 6 - DNA, 51 bp, synthetic construct
  source 1..51: mol_type = other DNA; note = a forward primer F1 of a primer combination for detecting the KASP marker associated with soybean resistance to a leaf-feeding pests
gaaggtgaccaagttcatgctgcacgaaccactattatctttttaatcttc

SEQ ID NO: 7 - DNA, 51 bp, synthetic construct
  source 1..51: mol_type = other DNA; note = a forward primer F2 of a primer combination for detecting the KASP marker associated with soybean resistance to a leaf-feeding pests
gaaggtcggagtcaacggattgcacgaaccactattatctttttaatcttt

SEQ ID NO: 8 - DNA, 24 bp, synthetic construct
  source 1..24: mol_type = other DNA; note = a reverse primer R of a primer combination for detecting the KASP marker associated with soybean resistance to a leaf-feeding pests
accagacaggatcaaagatacttt

SEQ ID NO: 9 - DNA, 23 bp, synthetic construct
  source 1..23: mol_type = other DNA; note = a forward primer for amplifying GmMYC3 gene from Glycine max KF NO. 1 and NN 1138-2
atggaggatttaatcatatctcc

SEQ ID NO: 10 - DNA, 21 bp, synthetic construct
  source 1..21: mol_type = other DNA; note = a reverse primer for amplifying GmMYC3 gene from Glycine max KF NO. 1 and NN 1138-2
tcactgatccaacctcatcag

SEQ ID NO: 11 - DNA, 21 bp, synthetic construct
  source 1..21: mol_type = other DNA; note = a forward primer sequence for fluorescence quantitative analysis of GmMYC3 gene
tggtctcgaggtcgatgtgaa

SEQ ID NO: 12 - DNA, 21 bp, synthetic construct
  source 1..21: mol_type = other DNA; note = a reverse primer sequence for fluorescence quantitative analysis of GmMYC3 gene
gacatgctagcgtgatgaacc

SEQ ID NO: 13 - DNA, 19 bp, synthetic construct
  source 1..19: mol_type = other DNA; note = a forward primer sequence for amplifying Tubulin gene of Glycine max
ggagttcacagaggcagag

SEQ ID NO: 14 - DNA, 20 bp, synthetic construct
  source 1..20: mol_type = other DNA; note = a reverse primer sequence for amplifying Tubulin gene of Glycine max
cacttacgcatcacatagca

SEQ ID NO: 15 - DNA, 21 bp, synthetic construct
  source 1..21: mol_type = other DNA; note = a reverse primer sequence for amplifying N-terminal of protein coded by GmMYC3 gene
cggagtctcgcgtccgagaat

SEQ ID NO: 16 - DNA, 21 bp, synthetic construct
  source 1..21: mol_type = other DNA; note = a forward primer sequence for amplifying C-terminal of protein coded by GmMYC3 gene
ataacccctccagatccggtt

SEQ ID NO: 17 - DNA, 20 bp, synthetic construct
  source 1..20: mol_type = other DNA; note = A primer sequence F1 for amplifying the target site
ggctctcatgaacttctttg

SEQ ID NO: 18 - DNA, 20 bp, synthetic construct
  source 1..20: mol_type = other DNA; note = a primer sequence R1 for amplifying the target site
caaagaagttcatgagagcc

SEQ ID NO: 19 - DNA, 20 bp, synthetic construct
  source 1..20: mol_type = other DNA; note = a primer sequence F2 for amplifying the target site
accaaagaggaagacgaaga

SEQ ID NO: 20 - DNA, 20 bp, synthetic construct
  source 1..20: mol_type = other DNA; note = a primer sequence R2 for amplifying the target site
tcttcgtcttcctctttggt

SEQ ID NO: 21 - DNA, 21 bp, synthetic construct
  source 1..21: mol_type = other DNA; note = a primer sequence F3 for amplifying the target site
acatgaacttctttgtgggaa

SEQ ID NO: 22 - DNA, 21 bp, synthetic construct
  source 1..21: mol_type = other DNA; note = a primer sequence R3 for amplifying the target site
ttcccacaaagaagttcatgt

SEQ ID NO: 23 - DNA, 23 bp, synthetic construct
  source 1..23: mol_type = other DNA; note = A forward primer sequence for overexpression soybean detection
gctcctacaaatgccatcattgc

SEQ ID NO: 24 - DNA, 23 bp, synthetic construct
  source 1..23: mol_type = other DNA; note = A reverse primer sequence for overexpression soybean detection
tcggttttctccctcttttcttc

SEQ ID NO: 25 - DNA, 20 bp, synthetic construct
  source 1..20: mol_type = other DNA; note = a forward primer sequence for testing gene-edited soybeans
atcttaatccggaacccacc

SEQ ID NO: 26 - DNA, 22 bp, synthetic construct
  source 1..22: mol_type = other DNA; note = a reverse primer sequence for testing gene-edited soybeans
acttaacgtgctgtacgaggtt

SEQ ID NO: 27 - DNA, 19 bp, synthetic construct
  source 1..19: mol_type = other DNA; note = a reverse primer sequence for vector construction
ctgatccaacctcatcaga